(19) United States
(12) Patent Application Publication
(10) Pub. No.: US 2026/0294995 A1
(43) Pub. Date: Oct. 1, 2026

(54) COMPOSITION FOR ORAL USE WITH HMG-COA REDUCTASE ENZYME INHIBITING ACTIVITY

(71) Applicant: DIFASS INTERNATIONAL S.P.A., Coriano (RN) (IT)

(72) Inventors: Alida Agostini, Coriano (RN) (IT); Francesca Benvenuti, Coriano (RN) (IT); Davide Giorgini, Savignano sul Rubicone (FC) (IT)

(73) Assignee: DIFASS INTERNATIONAL S.P.A., Coriano (RN) (IT)

(21) Appl. No.: 19/481,698

(22) PCT Filed: May 8, 2024
(86) PCT No.: PCT/IB2024/054487

§ 371 Date: Nov. 5, 2025

(30) Foreign Application Priority Data
May 9, 2023 (IT) 102023000009180

(51) Int. Cl. A61K 36/63; A61K 9/00; A61K 9/20; A61K 9/28; A61K 9/48; A61K 36/539; A61K 36/61; A61K 36/899; A61K 47/02; A61K 47/12; A61K 47/22; A61K 47/26; A61K 47/36

(52) U.S. Cl. CPC A61K 36/63; A61K 9/009; A61K 9/2009; A61K 9/2013; A61K 9/2027; A61K 9/2031; A61K 9/2054; A61K 9/2059; A61K 9/2095; A61K 9/28; A61K 9/485; A61K 9/4858; A61K 9/4866; A61K 9/4875; A61K 36/539; A61K 36/61; A61K 36/899; A61K 47/02; A61K 47/12; A61K 47/22; A61K 47/26; A61K 47/36; A61K 2236/333

(57) ABSTRACT

Disclosed are compositions comprising an Olea europaea extract and a Scutellaria baicalensis extract for use in the treatment of dyslipidemia as inhibitors of the HMG-CoA reductase enzyme.

The present invention relates to compositions comprising an Olea europaea extract and a Scutellaria baicalensis extract for use in the treatment of dyslipidaemia.

### PRIOR ART

2.6 million deaths worldwide are estimated to be attributable to hypercholesterolaemia. HMG-COA (3-hydroxy-3-methylglutaryl-CoA) reductase is an enzyme responsible for biosynthesis of cholesterol in the liver. Statins are the medicaments most widely used in the treatment of hypercholesterolaemia. Their action mechanism is competitive inhibition of said enzyme. Reduction of the intracellular cholesterol content gives rise to an increase in LDL receptors, leading to greater uptake and internalisation of cholesterol, thereby removing it from the bloodstream. Statins have a dose-dependent effect. Treatment with statins is not always well tolerated, especially due to their known possible side effects (hepatotoxicity and myopathy). Myopathy occurs in 10-13% of patients. The main reason for non-compliance with statin treatment is the muscle symptoms associated with statins (including the nocebo/drucebo effect). Moreover, in a fairly high percentage of patients (10-15% of low responders), treatment with statins does not enable adequate therapeutic targets to be achieved. In such cases, supplements (nutraceuticals) are prescribed in borderline situations and/or in primary prevention in addition to ezetimibe.

Especially in the last decade, various clinical trials have been conducted which demonstrated the activity against the blood cholesterol level of Olea europaea leaf and/or fruit extracts, excluding olive oil from this category as it consists almost entirely of fatty acids, with a much lower polyphenol content in proportion to the extracts, so that their composition is substantially different from said extracts. The studies do not always conclude that there is a significant beneficial effect, i.e. a significant reduction in “bad” (LDL) cholesterol.

The Lockyer et al. (2017) study concluded that daily administration of a leaf extract rich in polyphenols, amounting to 136 mg of oleuropein plus 6 mg of hydroxytyrosol a day for 6 weeks, reduced LDL by 6.37%.

The Stevens et al. (2021) study concluded that daily administration of a leaf extract rich in polyphenols, amounting to 83.5 mg of oleuropein a day for 8 weeks, had no effect on LDL.

The De Bock et al. (2013) study concluded that daily administration of a leaf extract rich in polyphenols, amounting to 51.1 mg of oleuropein plus 9.7 mg of hydroxytyrosol a day for 12 weeks, had no significant effect on LDL.

The Filip et al. (2015) study concluded that daily administration of a leaf extract rich in polyphenols, amounting to 100 mg of oleuropein a day for 12 weeks, reduced LDL by 20.73%.

The Araki et al. (2019) study concluded that daily administration of a leaf tea for 12 weeks to prediabetic patients significantly reduced LDL.

The Georgakouli et al. (2016) study concluded that daily administration of a yoghurt enriched with 50 mg of polyphenols from Olea europaea fruit for 2 weeks reduced LDL by 6.45%.

The Pais et al. (2016) study concluded that daily administration of a fruit extract, amounting to 50 mg or 100 mg of hydroxytyrosol a day for 11 days, did not reduce LDL.

In other clinical trials Olea europaea extracts were combined with other herbal extracts, and we will cite some of them by way of example; however, we have excluded those relating to combinations with statins or similar substances, such as monacolin K and monacolins in general present in fermentates with Monascus purpureus, such as red rice, because the latter are compounds with known anticholesterolemic activity, very similar to that of pharmacological statins.

The Sánchez Macarro et al. (2020) study concluded that daily administration of a combination of grapefruit and bitter orange extracts, amounting to 250-300 mg of flavanone glycosides and 175-200 mg of flavones, with a leaf extract rich in polyphenols, amounting to 85-90 mg of polyphenols (mainly oleuropein) a day for 8 weeks, reduced LDL by 6.10%.

The Wong et al. (2014) study concluded that daily administration of a combination of olive leaf extract, amounting to 160-240 mg of oleuropein, at least 150 mg of other polyphenols, green coffee bean extract, amounting to 90-100 mg of chlorogenic acid, 20-30 mg of caffeoylquinic acid and 100-110 mg of other polyphenols, and 300 mg of beetroot extract for 12 weeks, did not reduce LDL.

As regards the action mechanism, some in vitro studies demonstrate that oleuropein, a polyphenol present in olive extracts, reduces the activity of hydroxymethylglutaryl-CoA reductase (HMG-COA reductase), leading to a reduction in cholesterol synthesis in rat hepatocytes (Priore et al. 2014). When investigating the action mechanisms of olive polyphenols in an in vitro model with glial cells, the same research group found that hydroxytyrosol, although at a pharmacological concentration (25 μM), has a downregulating effect on the synthesis of fatty acids and cholesterol, reducing the activity of HMG-COA reductase by 16% (Priore et al. 2017). More recently, a phenol complex derived from olive oil processing waste, containing hydroxytyrosol, tyrosol and verbascoside as the main constituents, was studied on an in vitro model to evaluate its HMG-COA reductase inhibiting activity (Bartolomei et al. 2022): the results indicate that the extract reduces enzyme activity by 10±5.2%, 34.5±3.2%, 48.5±0.5%, 80.2±1.9% and 90±0.3% to 10.0, 50.0, 75.0, 100.0 and 500.0 μg/mL respectively.

Other compounds of plant origin have been studied and used in treatment, including Psidium guajava leaf and fruit extracts, resveratrol, Acacia catechu wood extracts, Scutellaria baicalensis root extracts, and policosanols extracted from sugar cane.

Psidium guajava (L.) is a plant belonging to the Myrtaceae family which grows in tropical regions such as India, Indonesia, Pakistan, Bangladesh and South America. Although the leaves of the plants are classed as agricultural waste, they are a rich source of bioactive compounds. The leaves are used in traditional medicine in Asian countries, mainly for their antihyperglycaemic effect (Kumar et al. 2021). A recent study on rats demonstrated that supplementing a high-fat diet with powdered Psidium guajava leaves (2.5%) improved dyslipidemia (Mamun et al. 2019). Another preclinical study on a diabetes model demonstrated that daily administration of 400 mg/kg of body weight of leaf extract reduces serum triglycerides, total cholesterol and LDL cholesterol, and increases HDL cholesterol (Tella et al. 2019).

Resveratrol is a polyphenol produced by various plants as a defense mechanism in response to the presence of parasites and other stressful situations such as climate. In humans, oral treatment with resveratrol seems to modulate the metabolism in various tissues; however, there is no evidence of the existence of specific receptors, especially associated with its absorption and pharmacokinetics. The majority of studies on resveratrol report cardioprotective effects and favourable effects in type 2 diabetes and Alzheimer's disease, as well as antithrombotic, antiosteoporotic and antimicrobial effects. A recent study demonstrated that a high daily dose of resveratrol (250 mg) for three months reduces the total cholesterol level in patients with metabolic syndrome (Batista-Jorge et al. 2020). A 2022 meta-analysis of randomised controlled trials concludes that resveratrol can be useful to reduce total cholesterol and LDL cholesterol levels, but also that the dose of resveratrol is an essential factor that influences the level of LDL-C(Cao et al. 2022).

Acacia catechu hardwood flavonoids have been used in traditional medicine, especially in Asia, for a variety of purposes: as anti-inflammatoires, antivirals, antibacterials and for cardiovascular health (Srivastava et al. 2010). A water-ethanol leaf extract was administered to diabetic rats for 30 days at concentrations of 200 mg/kg and 400 mg/kg. The extract reduced the total cholesterol, triglyceride, LDL and VLDL cholesterol levels, and increased the HDL cholesterol levels. (Jayabhara et al. 2018). A study on an animal model demonstrated that acacia polyphenol supplementation (2.5% and 5%) administered to mice with a high-fat diet generated a reduction in liver weight increase and triglyceride and cholesterol accumulation (Ikarashi et al. 2011).

Scutellaria baicalensis root is used in traditional Chinese medicine to treat inflammations, fever, coughs, dysentery and hypertension. Baicalin is the flavonoid found in the largest amount in Scutellaria baicalensis. Pharmacokinetics studies conducted with doses ranging from 100 mg to 2800 mg of baicalin did not indicate a proportionality in absorption depending on the oral dose (Li et al. 2014; Dong et al. 2021). Some indications in traditional medicine have been confirmed by recent pharmacological studies, such anti-tumoral, anti-inflammatory and anti-infective activity, and hepatoprotective and neuroprotective effects. The available literature indicates that most bioactivities are attributable to neo-clerodane flavonoids and diterpenoids. Although Scutellaria is used in clinical practice, research is still limited (Shen et al. 2021), as is know-how regarding its HMG-COA reductase-inhibiting potential. In an animal model, HMG-CoA reductase expression in mouse liver homogenate was significantly reduced in the group supplemented with Scutellaria baicalensis, compared with the non-supplemented groups (Lee et al. 2011). Screening of 98 different polyphenol compounds demonstrated the activity of baicalin and baicalin at the concentration of 10 μg/mL, finding inhibitory activity for both which was lower than with pravastatin (Son et al. 2018). The antihypercholesterolaemic activity of the straight-chain aliphatic alcohols known as policosanols, in particular those with an octacosanol content of at least 60%, has been evaluated in numerous clinical trials. There is no unanimous agreement on the effects of policosanols. A recent meta-analysis concluded that policosanols have no effect on the lipid profile (Osadnik et al. 2022), while a 2005 meta-analysis concluded that policosanols are more effective than sterols and stanols in reducing LDL cholesterol levels (Chen et al. 2005). A meta-analysis which examined data regarding a combination of red yeast rice, berberine and policosanols demonstrated that said combination is effective in improving the lipid profile, including cholesterol (Millán et al. 2016). As regards the action mechanism and involvement in endogenous cholesterol synthesis by HMG-COA reductase, a study on rats in 2019 demonstrated that policosanols do not affect mRNA expression of HMG-COA reductase, but reduce the activity of HMG-COA reductase during the development of hypercholesterolaemia induced by a high-fat, high-cholesterol diet. Moreover, the addition of policosanols stimulates phosphorylation of AMPK, which can effectively inhibit cholesterol levels by deactivating HMG-COA reductase (Nam et al. 2019).

WO 20212094948 describes compositions containing policosanols combined with Annurca apple and Olea europaea extracts useful for the treatment of dyslipidemia. The application does not report any synergistic effects between policosanols and Olea extract.

On the basis of the available literature, it is evident that the effect of reduction of the enzymatic activity of HMG-CoA reductase is dose-dependent. In view of the low bioavailability of polyphenols and the low content thereof in natural extracts, it would be desirable to find solutions that produce a beneficial effect for patients with hypercholesterolaemia, without the need to use high doses of polyphenols, which involve drawbacks in economic and safety terms (e.g. due to side effects, interference with ongoing pharmacological treatments, and overload on the excretory organs required to dispose of the residues of their catabolismit is even more important to find solutions allowing the use of small doses of bioactive compounds in view of the need for chronic treatment, because reduction of a cardiovascular risk factor such as LDL cholesterol must be implemented every day, and compliance with the treatment is essential to ensure that the lipid target and consequent cardiovascular risk control is achieved; such a solution would improve compliance, safety of use and economic sustainability.

### DESCRIPTION OF THE INVENTION

It has now been discovered that the combination of an Olea europaea fruit and leaf extract with a Scutellaria baicalensis root extract and possibly policosanols synergistically inhibits HMG-COA reductase.

One aspect of the invention therefore relates to compositions comprising or consisting of an Olea europaea fruit and leaf extract, a Scutellaria baicalensis root extract, and possibly policosanols as synergistic active ingredients for use in the treatment of dyslipidemia as inhibitors of the HMG-COA reductase enzyme.

The Olea europaea extract, obtained by extraction of leaves and fruit with mixtures of ethanol and water, preferably has a polyphenol content ranging between 5% and 95%, more preferably between 10% and 70%, and even more preferably between 15% and 50% of the weight of the extract. For example, the polyphenol content can be about 16% by weight. Scutellaria baicalensis extract is a water-alcohol root extract.

Scutellaria baicalensis root extract has a baicalin content ranging between 5% and 95%, preferably between 10% and 70%, and more preferably between 15% and 50% of the weight of the extract.

The Olea and Scutellaria extracts are known and available on the market.

According to preferred aspects, the compositions according to the invention comprise or consist of:
Olea europaea fruit and leaf extract, policosanols and a Scutellaria baicalensis root extract;
Olea europaea fruit and leaf extract and policosanols, preferably without other active ingredients such as plant extracts;
Olea europaea fruit and leaf extract, and a Scutellaria baicalensis root extract.

The weight ratio between the Olea europaea fruit and leaf extract and policosanols ranges between 1:1 and 50:1, preferably between 2:1 and 25:1; more preferably between 3:1 and 10:1.

The weight ratio between Olea europaea fruit and leaf extract and Scutellaria baicalensis root extract ranges between 0.1:1 and 5:1, preferably between 0.2:1 and 2:1, and more preferably between 1:1 and 0.5:1.

The dosage units typically fall into the following ranges:
5 mg to 300 mg of Olea europaea fruit and leaf extract; preferably 20 mg to 150 mg; more preferably 30 mg to 100 mg;
2 mg to 30 mg of policosanols; preferably 10 mg to 25 mg; more preferably 15 mg to 20 mg;
5 mg to 300 mg of Scutellaria baicalensis root extract; preferably 20 mg to 200 mg; more preferably 50 mg to 150 mg.

The compositions according to the invention can also include as a further active ingredient 10 mg to 400 mg of Psidium guajava leaf and/or fruit extract per dosage unit; preferably 20 mg to 200 mg; more preferably 50 mg to 150 mg.

Other herbal derivatives may also be present in the form of extracts or powders, fermentates, probiotics, yeasts, bacterial lysates, postbiotics, prebiotics, oils, essences, polyphenols, bioflavonoids, berberine, plant sterols, plant stanols, fungi or seaweed in the form of extracts or powders, lipoic acid, glutathione (GSH), melatonin, resveratrol, GABA, coenzyme Q10, choline, amino acids, vitamins or minerals.

The compositions according to the invention are formulated as dietary, nutraceutical or pharmaceutical preparations using conventional techniques and excipients.

Formulation examples comprise swallowable tablets, chewable tablets, hard capsules, soft capsules, granulates and powders in jars or sachets to be reconstituted in water or taken directly in the mouth, solutions, suspensions, liquid, orosoluble or orodispersible stick packs, vials and two-phase ampoules.

To demonstrate the synergistic effects characteristic of the compositions according to the invention, the inhibitory effect of various natural extracts on the HMG-COA reductase enzyme was evaluated: Olea europaea leaf and fruit extract (total polyphenol content 16%; plant/extract ratio=8:1), policosanols extracted from sugar cane (total policosanol content ≥98%, octacosanol content ≥60%), resveratrol extract from Polygonum cuspidatum (trans-resveratrol content over 98%), Scutellaria baicalensis G. root extract (baicalin content ≥30%), Acacia catechu woody part extract (catechin and epicatechin content ≥21%), Punica granatum pericarp extract (ellagic acid content ≥40%), Psidium guajava leaf extract (punicalagin content ≥20%; plant/extract ratios=4:1), Psidium guajava fruit extract (punicalagin content ≥20%).

The screening was performed with an analysis kit that evaluates inhibition of HMG-CoA reductase. The assay was based on spectrophotometric measurement of the reduction of absorbance at 340 nm, which represents oxidation of NADPH by the catalytic subunit of HMG-CoA reductase in the presence of the HMG-COA substrate. The reaction is as follows: HMG-CoA+2NADPH+2H⁺ [symbol] mevalonate+2NADP⁺+CoA-SH. Modulation of the activity of the enzyme, and in particular its inhibition, was calculated as % variation relative to the activity measured under the control condition, i.e. in the absence of any inhibitor. The biochemical system was verified with the use of a positive control, pravastatin, at the doses of 212.5 mg (100 mM), 80 mg, 40 mg and 20 mg.

A first test session was conducted with pravastatin as positive control at the theoretical concentration calculated as administration dose in the systemic circulation (5 L, mean blood volume of an adult man weighing about 70 kg);

Table 1 presents the activity data of the enzyme in the presence of pravastatin

TABLE 1

| | Percentage inhibition HMGR |
|---|---|
| Pravastatin 212.5 mg | 100.0% |
| Pravastatin 80 mg | 85.9% |
| Pravastatin 40 mg | 68.9% |
| Pravastatin 20 mg | 56.3% |

Each test was conducted in triplicate, and the compounds were tested at different concentrations.

Various sessions were conducted to evaluate the inhibitory activity of each ingredient, assuming the following oral administration doses:
Policosanols 10 mg
Psidium guajava leaf extract 100 mg
Psidium guajava fruit extract 100 mg
Punica granatum extract 100 mg
Scutellaria baicalensis extract 100 mg
Resveratrol 10 mg
Acacia catechu extract 100 mg
Olea europaea leaf and fruit extract 100 mg

A first test session (Test 1) was conducted on a 1:200 dilution of the theoretical concentration of all ingredients at the administration dose in the systemic circulation (5 L, mean blood volume of an adult man weighing about 70 kg).

Table 2 presents the data regarding the activity of the enzyme in the presence of the various ingredients under the conditions tested.

TABLE 2

| | Concentration | Percentage |
|---|---|---|
| Ingredient | μg/ml | inhibition HMGR |
| Policosanols | 0.0102 | 31.8% |
| Psidium guajava leaf extract | 0.1000 | 45.9% |
| Psidium guajava fruit extract | 0.1000 | 36.4% |
| Punica granatum extract | 0.1000 | 3.7% |
| Scutellaria baicalensis extract | 0.1000 | 23.6% |
| Resveratrol | 0.0102 | 0.0% |
| Acacia catechu extract | 0.1000 | 0.0% |
| Olea europaea leaf and fruit extract | 0.1000 | 0.0% |

As described above, the combinations of the ingredients which performed best in Test 1 at a 1:200 dilution were investigated with the aim of finding solutions that produce a beneficial effect for patients with hypercholesterolaemia, with no need to use large doses of polyphenols. We decided to include among the combinations the total extract of Olea europaea, although it has no inhibitory activity at the 1:200 dilution, because it is unanimously recognised as a valuable part of the Mediterranean diet, with numerous health-giving properties.

Surprisingly, an unexpected synergy emerged between the Olea extract, which has no inhibitory activity (see Test 1), and policosanols, with +40.9% compared with the sum of the individual ingredients, and Scutellaria baicalensis root extract, with +44.1% compared with the sum of the individual ingredients.

Even more surprisingly, a synergy emerged between the Olea europaea fruit and leaf extract, policosanols and Scutellaria baicalensis root extract. In fact, the percentage inhibition recorded by the mixture of the three ingredients is greater not only than the sum of the individual ingredients (+42.2%), but also than the sum of the combination of two ingredients only (Olea plus policosanols and Olea plus Scutellaria), for which synergistic effects had already been found.

Not only did no interference between the ingredients emerge, an expected event found with the other combinations, but the result was significantly greater than the sum of the individual results for inhibition of the HMGR enzyme. See Table 3.

TABLE 3

| | % inhibition | |
|---|---|---|
| Ingredient (1:200) | HMGR | % Δ |
| Olea europaea + | 44.8% | +40.9% vs (Olea europaea + policosanols) |
| Policosanols | | |
| Olea europaea + | 34.0% | +44.1% vs (Olea europaea + Scutellaria |
| Scutellaria baicalensis | | baicalensis) |
| Olea europaea + | 78.8% | +42.2% vs (Olea europaea + policosanols + |
| policosanols + | | Scutellaria baicalensis) |
| Scutellaria baicalensis | | +15.2% vs [(Olea europaea + policosanols) + |
| | | Scutellaria baicalensis] |
| | | +19.8% vs [(Olea europaea + Scutellaria |
| | | baicalensis) + policosanols] |

The percentages of inhibition of the HMGR enzyme by other combinations of Olea europaea and the best-performing ingredients in test 1 are set out below by way of example, demonstrating the inefficiency of other combinations. See Table 4.

TABLE 4

| | % inhibition | |
|---|---|---|
| Ingredient (1:200) | HMGR | % Δ |
| Olea europaea + | 62.9% | −19.0% vs (Olea europaea + |
| policosanols + | | policosanols + |
| Psidium guajava leaf extract | | Psidium guajava leaves) |
| | | −30.7% vs (Olea europaea + |
| | | policosanols + |
| | | Psidium guajava leaves] |
| Olea europaea + | 47.1% | −30.9% vs (Olea europaea + policosanols + |
| policosanols + | | Psidium guajava fruit) |
| Psidium guajava fruit extract | | −42.0% vs (Olea europaea + |
| | | policosanols + Psidium guajava fruit] |

To establish the actual synergy between Olea europaea, policosanols and Scutellaria baicalensis, doses and weight ratios between them different from those described above were tested, maintaining the dilution (1:200). See Table 5.

TABLE 5

| | | % inhibition | |
|---|---|---|---|
| Ingredient | Oral dose | HMGR | % Δ |
| Policosanols | 15 mg | 46.5% | |
| Olea europaea + | 50 mg-15 mg | 56.5% | +21.5% vs (Olea europaea + |
| policosanols | | | policosanols 15 mg) |
| Olea europaea + | 50 mg-15 mg- | 83.1% | +3.7% vs [(policosanols15 + |
| policosanols + | 100 mg | | Olea europaea50*) + Scutellaria |
| Scutellaria | | | baicalensis100] |
| baicalensis | | | +18.5% vs (policosanols15 + |
| | | | Olea europaea50 + Scutellaria |
| | | | baicalensis100) |
| Olea europaea + | 50 mg-100 mg | 27.1% | +14.8% vs (Olea europaea50 + |
| Scutellaria | | | Scutellaria baicalensis100) |
| baicalensis | | | |
| Olea europaea + | 100 mg-100 mg | 34.0% | +44.1% vs (Olea europaea100 + |
| Scutellaria | | | Scutellaria baicalensis100) |
| baicalensis | | | |
| Policosanols + | 15 mg-100 mg- | 78.7% | +12.3% vs |
| Olea europaea + | 100 mg | | (policosanols15 + Olea100 + |
| Scutellaria | | | Scutellaria baicalensis100) |
| baicalensis | | | |

Examples of formulations according to the invention, obtained with conventional techniques and excipients, are set out below.

### Examples 1-3: Hard Capsules

### Example 1

| | mg/capsule | |
|---|---|---|
| Olea europaea leaf and fruit extract | 50.0 | mg |
| Policosanols | 15.0 | mg |
| Scutellaria baicalensis extract | 100.0 | mg |
| Magnesium stearate | 12.0 | mg |
| Silicon dioxide | 8.0 | mg |
| Dibasic calcium phosphate | 216.0 | mg |
| Size “0” capsule | 99.0 | mg |
| TOTAL | 500.0 | mg |

### Example 2

| | mg/capsule | |
|---|---|---|
| Olea europaea leaf and fruit extract | 50.0 | mg |
| Policosanols | 15.0 | mg |
| Scutellaria baicalensis extract | 100.0 | mg |
| Psidium guajava leaf extract | 100.0 | mg |
| Magnesium stearate | 8.0 | mg |
| Silicon dioxide | 8.0 | mg |
| Dibasic calcium phosphate | 120.0 | mg |
| Size “0” capsule | 99.0 | mg |
| TOTAL | 500.0 | mg |

### Example 3

| | mg/capsule | |
|---|---|---|
| Olea europaea leaf and fruit extract | 50.0 | mg |
| Policosanols | 15.0 | mg |
| Scutellaria baicalensis extract | 100.0 | mg |
| Green tea extract | 70.0 | mg |
| Vitamin E acetate | 44.5 | mg |
| Vitamin B3 | 27.0 | mg |
| Pyridoxine hydrochloride | 2.85 | mg |
| Vitamin B12 0.1% | 1.15 | mg |
| Folic acid | 0.40 | mg |
| Silicon dioxide | 8.0 | mg |
| Dibasic calcium phosphate | 82.1 | mg |
| Size “0” capsule | 99.0 | mg |
| TOTAL | 500.0 | mg |

### Examples 4-5: Tablets/Coated Tablets

The tablets can be prepared by direct compression of the ingredients or by compression of granular forms thereof (dry or wet granulation). The ingredients can be advantageously divided between two or more layers of the tablet with different ingredient release kinetics.

### Example 4

| | mg/tablet | |
|---|---|---|
| Olea europaea leaf and fruit extract | 50.00 | mg |
| Policosanols | 15.00 | mg |
| Phytosterols | 505.05 | mg |
| Vitamin B3 | 27.00 | mg |
| Microcrystalline cellulose | 111.52 | mg |
| Maltodextrins | 28.00 | mg |
| Dicalcium phosphate | 116.44 | mg |
| Crosslinked sodium carboxymethylcellulose | 25.00 | mg |
| Polyvinylpolypyrrolidone | 25.00 | mg |
| Magnesium stearate | 8.00 | mg |
| Silicon dioxide | 5.00 | mg |
| Hydroxypropyl methylcellulose | 21.52 | mg |
| Polyvinylpyrrolidone | 5.60 | mg |
| Polyethylene glycol | 0.90 | mg |
| Calcium carbonate | 4.97 | mg |
| Iron oxide | 1.00 | mg |
| TOTAL | 950.00 | mg |

### Example 5

mg/tablet

| | | |
|---|---|---|
| Olea europaea leaf and fruit extract | 50.00 | mg |
| Policosanols | 15.00 | mg |
| Scutellaria baicalensis extract | 100.00 | mg |
| Berberine granules | 588.24 | mg |
| Vitamin E acetate | 77.60 | mg |
| Vitamin B3 | 35.10 | mg |
| Pyridoxine hydrochloride | 3.13 | mg |
| Vitamin B12 0.1% | 1.40 | mg |
| Folic acid | 0.45 | mg |
| Microcrystalline cellulose | 184.49 | mg |
| Dibasic calcium phosphate | 115.59 | mg |
| Silicon dioxide | 8.00 | mg |
| Crosslinked sodium carboxymethylcellulose | 13.00 | mg |
| Magnesium stearate | 8.00 | mg |
| TOTAL | 1,200.00 | mg |

### Example 6: Sachets/Stick Packs Containing Water-Dispersible and/or Orodispersible Powders and/or Granulates

| | mg/sachet | |
|---|---|---|
| Olea europaea leaf and fruit extract | 100.00 | mg |
| Policosanols | 15.00 | mg |
| Scutellaria baicalensis extract | 100.00 | mg |
| Psidium guajava leaf extract | 100.00 | mg |
| Vitamin B6 | 0.70 | mg |
| Folic acid | 0.10 | mg |
| Vitamin B12 | 1.25 | μg |
| Vitamin E | 6.00 | mg |
| Vitamin B1 | 0.55 | mg |
| Maltodextrin | 1,484.65 | mg |
| Silicon dioxide | 20.00 | mg |
| Flavouring | 60.00 | mg |
| Citric acid | 80.00 | mg |
| Xanthan gum | 20.00 | mg |
| Sucralose | 13.00 | mg |
| TOTAL | 2,000.00 | mg |

### Example 7: Soft Capsules

Before the capsule-filling process begins, two preparatory processes are usually conducted simultaneously but separately, leading to the formation of the two parts of a soft capsule, i.e. manufacture of (1) the gel mass and (2) the matrix which acts as filler for said capsules.

| | % w/w |
|---|---|
| Olea europaea leaf and fruit extract | 2.66% |
| Policosanols | 0.80% |
| Scutellaria baicalensis extract | 5.32% |
| Other active ingredients | 54.61% |
| Gelatin | 18.74% |
| Glycerin | 7.25% |
| Yellow beeswax | 5.33% |
| Glyceryl monostearate | 2.67% |
| Sunflower lecithin | 2.40% |
| Iron oxides | 0.22% |
| TOTAL | 100.00% |

### Example 8: Solutions/Suspensions

| | % w/v |
|---|---|
| Olea europaea leaf and fruit extract | 0.77% |
| Policosanols | 0.08% |
| Scutellaria baicalensis extract | 0.77% |
| Other active ingredients | 6.84% |
| Glycerol | 36.44% |
| Sorbitol | 5.12% |
| Flavouring | 1.82% |
| Xanthan gum | 0.69% |
| Citric acid | 0.60% |
| Preservatives | 0.23% |
| Sucrose ester | 0.12% |
| Sweeteners | 0.03% |
| Water | q.s. 100% |
| TOTAL | 100% |

### BIBLIOGRAPHY

Bartolomei M, Bollati C, Li J, Arnoldi A, Lammi C. Assessment of the Cholesterol-Lowering Effect of MOMAST®: Biochemical and Cellular Studies. Nutrients 2022, 14, 493.
Batista-Jorge, G. C.; Barcala-Jorge, A. S.; Silveira, M. F.; Lelis, D. F.; Andrade, J. M. O.; de Paula, A. M. B.; Guimarães, A. L. S.; Santos, S. H. S. Oral Resveratrol Supplementation Improves Metabolic Syndrome Features in Obese Patients Submitted to a Lifestyle Changing Program. Life Sci. 2020, 256, 117962.
Cao, X.; Liao, W.; Xia, H.; Wang, S.; Sun, G. The Effect of Resveratrol on Blood Lipid Profile: A Dose-Response Meta-Analysis of Randomized Controlled Trials. Nutrients 2022, 14, 3755.
Chen, J. T.; Wesley, R.; Shamburek, R. D.; Pucino, F.; Csako, G. Meta-analysis of natural therapies for hyperlipidemia: Plant sterols and stanols versus policosanol. Pharmacotherapy 2005, 25, 171-183.
de Bock M, Derraik J G, Brennan C M et al (2013) Olive (Olea europaea L.) leaf polyphenols improve insulin sensitivity in middle-aged overweight men: a randomized, placebo-controlled, crossover trial. PLOS One 8: e57622.
Dong, R.; Li, L.; Gao, H.; Lou, K.; Luo, H.; Hao, S.; Yuan, J.; Liu, Z. Safety, tolerability, pharmacokinetics, and food effect of baicalin tablets in healthy Chinese subjects: A single-center, randomized, double-blind, placebo-controlled, single-dose phase I study. J. Ethnopharmacol. 2021, 274, 114052.
Filip R, Possemiers S, Heyerick A, Pinheiro I, Raszewski G, Davicco M J, et al. Twelve-month consumption of a polyphenol extract from olive (Olea europaea) in a double blind, randomized trial increases serum total osteocalcin levels and improves serum lipid profiles in postmenopausal women with osteopenia. Evid Based Complement and Altern Med. 2015; 19:77-86.
Ikarashi, N.; Toda, T.; Okaniwa, T.; Ito, K.; Ochiai, W.; Sugiyama, K. Anti-obesity and anti-diabetic effects of acacia polyphenol in obese diabetic KKAy mice fed high-fat diet. Evid.-Based Complement. Altern. Med. 2011, 2011, 952031.
Jayabharathi, V.; Ramesh, B. Evaluation of antihyperglycemic and antihyperlipidemic activities of Acacia catechu (l.f) wild leaf extract in Wistar albino rats. Int. J. Green Pharm. 2018, 12, 175-181.
Kumar M, Tomar M, Amarowicz R, Saurabh V, Nair M S, Maheshwari C, et al. Leaves: Nutritional Composition, Phytochemical Profile, and Health Promoting Bioactivities. Foods. 2021; 10:752.
Lee I S, Park S, Park K, Choue R. Hepatoprotective activity of Scutellariae radix extract in mice fed a high fat diet with chronic alcohol exposure. Phytother Res. 2011; 25:1348-1353.
Li, M.; Shi, A.; Pang, H.; Xue, W.; Li, Y.; Cao, G.; Yan, B.; Dong, F.; Li, K.; Xiao, W.; et al. Safety, tolerability, and pharmacokinetics of a single ascending dose of baicalin chewable tablets in healthy subjects. J. Ethnopharmacol. 2014, 156, 210-215.
Lockyer, S.; Rowland, I.; Spencer, J. P. E.; Yaqoob, P.; Stonehouse, W. Impact of Phenolic-Rich Olive Leaf Extract on Blood Pressure, Plasma Lipids and Inflammatory Markers: A Randomised Controlled Trial. Eur. J. Nutr. 2017, 56, 1421-1432.
Mamun, M., Al, A., Faruk, M., Rahman, M., Nahar, K., Kabir, F., Alam, M. A., & Subhan, N. (2019). High carbohydrate high fat diet induced hepatic steatosis and dyslipidemia were ameliorated by Psidium guajava Leaf powder supplementation in rats. Evidence-Based Complementary and Alternative Medicine, 2019, 2019-2012.
Millán J, Cicero A F G, Torres F, et al. Effects of a nutraceutical combination containing berberine (BRB), policosanol, and red yeast rice (RyR), on lipid profile in hypercholesterolemic patients: a metaanalysis of randomised controlled trials. Clin Investig Arterioscler 2016; 28:178-87.
Nam, D. E.; Yun, J. M.; Kim, D.; Kim, O. K. Policosanol Attenuates Cholesterol Synthesis via AMPK Activation in Hypercholesterolemic Rats. J. Med. Food 2019, 22, 1110-1117.
Osadnik T, Goławski M, Lewandowski P, Morze J, Osadnik K, Pawlas N, Lejawa M, Jakubiak G K, Mazur A, Schwingschackl L, Gąsior M, Banach M. A network meta-analysis on the comparative effect of nutraceuticals on lipid profile in adults. Pharmacological Research 183 (2022) 106402.
Priore P, Siculella L, Gnoni G V. Extra virgin olive oil phenols down-regulate lipid synthesis in primary-cultured rat-hepatocytes. J Nutr Biochem. 2014; 25:683-91.
Priore P, Gnoni A, Natali F, Testini M, Gnoni G V, Siculella L, Damiano F. Oleic Acid and Hydroxytyrosol Inhibit Cholesterol and Fatty Acid Synthesis in C6 Glioma Cells. Oxidative Medicine and Cellular Longevity Volume 2017, Article ID 9076052, 10 pages.
Shen, J.; Li, P.; Liu, S.; Liu, Q.; Li, Y.; Sun, Y.; He, C.; Xiao, P. Traditional uses, ten-years research progress on phytochemistry and pharmacology, and clinical studies of the genus Scutellaria. J. Ethnopharmacol. 2021, 265, 113198.
Son K H, Lee J Y, Lee J S, Kang S S, Sohn H Y, Kwon C S. Screening of Flavonoid Compounds with HMG-COA Reductase Inhibitory Activities. Journal of Life Science 2018 Vol. 28. No. 2. 247~256.
Srivastava P S, Mishra A, Bhatia V, Narender T, Srivastava A K. Acacia catechu hard wood: potential anti-diabetic cum anti-dyslipidemic. Med Chem Res. 2010. DOI 10.1007/s00044-010-9479-y.
Stevens Y, Winkens B, Jonkers D, Masclee A. The effect of olive leaf extract on cardiovascular health markers: a randomized placebo-controlled clinical trial. Eur J Nutr. 2021; 60:2111-20.
Tella T, Masola B, Mukaratirwa S. The effect of Psidium guajava aqueous leaf extract on liver glycogen enzymes, hormone sensitive lipase and serum lipid profile in diabetic rats. Biomedicine & Pharmacotherapy, vol. 109, pp. 2441-2446, 2019.

1. A method of treating dyslipidaemia in a patient in need thereof, said method comprising
administering to said patient a pharmaceutical effective amount of a composition Compositions comprising or consisting of an Olea europaea fruit and leaf extract and a Scutellaria baicalensis root extract for use in the treatment of dyslipidemia as inhibitors of the HMG-COA reductase enzyme.

2. The method according to claim 1, wherein the Olea europaea fruit and leaf extract has a polyphenol content ranging between 5% and 95%, preferably between 10% and 70%, and more preferably between 15% and 50% of the weight of the extract.

3. The method according to claim 1, wherein the Scutellaria baicalensis root extract has a baicalin content ranging between 5% and 95%, preferably between 10% and 70%, and more preferably between 15% and 50% of the weight of the extract.

4. The method according to claim 1, wherein the composition further comprises policosanols.

5. The method Compositions for use according to claim 1, said composition not containing any other plant extracts.

6. The method according to claim 1, wherein the weight ratio of Olea europaea fruit and leaf extract to polycosanols ranges between 1:1 and 50:1, preferably between 2:1 and 25:1; and more preferably between 3:1 and 10:1.

7. The method according to claim 1, wherein the weight ratio between Olea europaea fruit and leaf extract and Scutellaria baicalensis root extract ranges between 0.1:1 and 5:1, preferably between 0.2:1 and 2:1, and more preferably between 1:1 and 0.5:1.

8. The method according to claim 1, said composition comprising 5 mg to 300 mg of Olea europaea fruit and leaf extract per dosage unit; preferably 20 mg to 150 mg; and more preferably 30 mg to 100 mg.

9. The method according to claim 1, said composition comprising 2 mg to 30 mg of polycosanols per dosage unit; preferably 10 mg to 25 mg; and more preferably 15 mg to 20 mg.

10. The method according to claim 1, said composition comprising 5 mg to 300 mg of Scutellaria baicalensis root extract per dosage unit; preferably 20 mg to 200 mg; and more preferably 50 mg to 150 mg.

11. The method according to claim 1, said composition, further comprising 10 mg to 400 mg of Psidium guajava leaf and/or fruit extract per dosage unit; preferably 20 mg to 200 mg; and more preferably 50 mg to 150 mg.

12. The method according to claim 1, said composition, further comprising one or more ingredients selected from herbal derivatives in the form of extracts or powders, fermentates, probiotics, yeasts, bacterial lysates, postbiotics, prebiotics, oils, essences, polyphenols, bioflavonoids, berberine, plant sterols, plant stanols, fungi in the form of extracts or powders, algae in the form of extracts or powders, lipoic acid, glutathione, melatonin, resveratrol, GABA, coenzyme Q10, choline, amino acids, vitamins and minerals.

13. The method according to claim 1, wherein said composition is in the form of swallowable tablets, chewable tablets, hard capsules, soft capsules, granules or powders in a jar or sachet to be reconstituted in water or taken directly in the mouth, solutions, suspensions, liquid, orosoluble or orodispersible stick packs, vials or biphasic ampoules.
